(19) United States
(12) Patent Application Publication
(10) Pub. No.: US 2025/0333747 A1
(43) Pub. Date: Oct. 30, 2025

(54) 5'-UTR WITH IMPROVED TRANSLATION EFFICIENCY, A SYNTHETIC NUCLEIC ACID MOLECULE INCLUDING THE SAME, AND A VACCINE OR THERAPEUTIC COMPOSITION INCLUDING THE SAME

(71) Applicants: GREEN CROSS CORPORATION, Gyeonggi-do (KR); MOGAM INSTITUTE FOR BIOMEDICAL RESEARCH, Seoul (KR)

(72) Inventors: Min-Kyung SHIN, Daejeon (KR); Hongseok HA, Daejeon (KR); Joori PARK, Daejeon (KR); Sena LEE, Daejeon (KR); Yoon Ki KIM, Daejeon (KR); Jaesung JUNG, Gyeonggi-do (KR); Yoon Suk LEE, Gyeonggi-do (KR); Hyokyoung KWON, Gyeonggi-do (KR); Tae-Hee KIM, Gyeonggi-do (KR); Yeomin YUN, Gyeonggi-do (KR)

(21) Appl. No.: 18/715,815

(22) PCT Filed: Dec. 2, 2022
(86) PCT No.: PCT/KR2022/019491

§ 371 Date: Jun. 3, 2024

(30) Foreign Application Priority Data
Dec. 3, 2021 (KR) 10-2021-0172306

(51) Int. Cl. C12N 15/67; A61K 39/00

(52) U.S. Cl. CPC C12N 15/67; A61K 39/00; A61K 2039/53; A61K 2039/55572

(57) ABSTRACT

Disclosed are a synthetic nucleic acid molecule including 5′-UTR with improved translation efficiency and a vaccine/therapeutic composition including the same, and more particularly, a 5′-UTR polynucleotide that is imparted with improved translation efficiency based on the specific motif thereof, a synthetic nucleic acid molecule including the same and a vaccine/therapeutic composition including the synthetic nucleic acid molecule. The 5′-UTR polynucleotide effectively induces expression of target proteins due to improved translation efficiency thereof and thus is useful for various RNA-based applications, for example, vaccines, in vivo/ex vivo gene therapy, etc.

### CROSS-REFERENCE TO RELATED APPLICATIONS

This application is a U.S. national phase under 35 U.S.C. §371 of International Patent Application No. PCT/KR2022/019491 filed Dec. 2, 2022, which in turn claims priority under 35 U.S.C. §119 of Korean Patent Application No. 10-2021-0172306 filed Dec. 3, 2021. The disclosures of all such applications are hereby incorporated herein by reference in their respective entireties, for all purposes.

### REFERENCE TO SEQUENCE LISTING

This application includes an electronically submitted sequence listing in .xml format. The .xml file contains a sequence listing entitled “724_SeqListing.xml” created on Jun. 2, 2024 and is 35,007 bytes in size. The sequence listing contained in this .xml file is part of the specification and is hereby incorporated by reference herein in its entirety.

### Field of the Invention

The present invention relates to a synthetic nucleic acid molecule including 5′-UTR with improved translation efficiency and a vaccine/therapeutic composition including the same, and more particularly, to a 5′-UTR polynucleotide that is imparted with improved translation efficiency based on the specific motif thereof, a synthetic nucleic acid molecule including the same and a vaccine/therapeutic composition including the synthetic nucleic acid molecule.

### Description of the Related Art

It has been reported that the untranslated region (UTR) in mRNA plays a pivotal role in the regulation of both stability and translation of mRNA. UTR is known to affect translation initiation, elongation, and termination as well as mRNA stabilization and intracellular localization through its interaction with RNA binding proteins (Jackson R J, et al., Nat Rev Mol Cell Biol. Vol. 11(2), pp. 113-127, 2010). Depending on the specific motif in the UTR, this may increase or decrease the mRNA turnover (Barrett L W, et al., Cell Mol Life Sci. Vol. 69(21), pp. 3613-34, 2012). Recently, data on mRNA half-life and the corresponding UTR sequence have been published (Hoen P A, et al., Nucleic Acids Res. Vol. 39(2), pp. 556-566, 2012).

UTRs refer to sections of an mRNA molecule in the upstream of the start codon and downstream of the stop codon of the mRNA, i.e., untranslated sequences. These regions are transcribed along with the coding regions and therefore are exons as they are present in mature mRNA. The UTR upstream of the start codon of the mRNA is referred to as “5′ UTR” and, once transcribed, in particular, possesses so-called “Kozak sequence”, along with the sequence corresponding to the (remaining 3′) portion of the promoter.

A Kozak consensus sequence (Kozak consensus or Kozak sequence) is known to be found in eukaryotic mRNA and has a consensus sequence of (gcc)gccRccAUGG. The Kozak consensus sequence plays a major role in the initiation of the translation process. The sequence is named after Marilyn Kozak who discovered the significance thereof. This sequence in the mRNA molecule is recognized by the ribosome at the translation start site, from which the protein is encoded by the mRNA molecule. The ribosome requires this sequence or a possible variant thereof to initiate translation.

The sequence is identified by the notation (gcc) gccRccAUGG, which summarizes data analyzed by Kozak from a wide variety of sources (about 699 in all) as follows: a lower-case letter denotes the most common base at a position where the base can nevertheless vary; upper-case letters indicate highly conserved bases, i.e. the ‘AUGG’ sequence is constant or rarely, if ever, changes; “R” indicates that a purine (adenine or guanine) is always observed at this position (with adenine being more frequent according to Kozak); and the sequence in parentheses (gcc) is of uncertain significance.

A number of means and methods (U.S. Pat. No. 10,080,809, US 2018-0353618, US 2019-0144883) to increase the stability of mRNA, reduce the immunogenic response triggered by mRNA administered to cells or organisms, and increase expression efficiency (i.e., transcription and/or translation efficiency) have been published. However, in particular, there is still a need for improvement for additional or alternative means to increase expression efficiency (i.e. transcription and/or translation efficiency). This is because expression efficiency is an essential parameter for anticipated medical applications because it, for example, determines the administration and interval of administration of mRNA drugs and ultimately determines the bioavailability of the final products, i.e., the encoded peptides or proteins. At the same time, there is still a need to further reduce the production cost of the mRNA drugs, increase the yield of the produced mRNA molecules, and increase the available space for the coding region encoding the actual transgenes, i.e., the polypeptides of interest, within the resulting mRNA molecules.

Meanwhile, genetic vaccines have been developed since it was reported that if DNA and RNA encoding target genes are directly injected into animals, the target genes are expressed in living animals, and immunity can be established by this expression (Wolff J A et al. Science, 247:1465-8, 1990).

Genetic vaccination elicits a desired immune response against selected antigens, such as characteristic components of bacterial surfaces, viral particles, and tumor antigens. Generally, vaccination is one of the pivotal achievements of modern medicine. However, effective vaccines are currently available only for a limited number of diseases. Thus, infections that cannot be prevented by vaccination still affect millions of people annually.

DNA and RNA may be used as nucleic acid molecules for gene administration in gene therapy or genetic vaccination and DNA is known to be relatively stable and tractable compared to RNA. However, DNA may cause a potential risk if the DNA-fragment administered to the genome of patients is inserted at an undesired location, resulting in damage to the gene. Further, undesired anti-DNA antibodies may occur and another problem is that the expression level of peptides or proteins expressed by DNA administration and subsequent transcription/translation is limited. The presence or absence of a specific transcription factor that regulates DNA transcription has a major impact on the expression level of the administered DNA, and in the absence of the specific transcription factor, a sufficient amount of RNA is not produced by DNA transcription and as a result, the level of the peptide or protein that is translated and produced is also limited.

Meanwhile, when RNA is used as the means for gene administration, RNA does not require transcription and thus is capable of synthesizing proteins directly in the cytoplasm without having to enter the nucleus like DNA, thus having no risk of interfering with cell chromosomes and causing undesired gene damage. In addition, RNA does not induce long-term genetic modification due to short half-life compared to DNA (Sayour E J, et al., J Immunother Cancer Vol. 3, 13, 2015). When a general RNA vaccine is delivered into cells, it is activated only for a short time to express the target protein, and is then destroyed by an enzymatic reaction within a few days, and a specific immune response to the expressed target antigen (protein) remains.

In addition, when RNA is used as the means for gene administration, it acts only when it passes through the cell membrane, without the need to pass through the nuclear membrane. The target protein may be expressed in the same amount as in DNA in spite of using a smaller amount than DNA. In addition, RNA itself has immune adjuvanticity and thus exhibits the same immune effect even when administered in a small amount compared to DNA. By using RNA instead of DNA for genetic vaccination, the risk of unwanted genomic integration and anti-DNA antibody generation is minimized or avoided. However, RNA is considered a fairly unstable molecule that may be readily degraded by ubiquitous RNases.

Although great advances have been made in the past few years, inefficient translation of mRNA due to premature degradation of antigen or inefficient release of mRNA from cells as an efficient mRNA vaccination method capable of inducing an adaptive immune response still remains in the art. Furthermore, there is an increasing need to reduce the dose of mRNA vaccines in order to reduce the concerns of potential safety and to make the vaccine affordable in the third world.

There are many problems associated with the delivery of nucleic acids to induce a desired response in a biological system. Nucleic acid-based therapeutics such as vaccines have great potential, but there is still a need for more effective delivery of nucleic acids to appropriate sites within cells or organisms to realize this potential.

However, the therapeutic and prophylactic applications of nucleic acids currently face two problems. First, free RNA is susceptible to nuclease digestion in plasma. Second, free RNA has a limited ability to access intracellular compartments where the associated translation agent resides. Lipid nanoparticles produced from cationic lipids and other lipid components such as neutral lipids, cholesterol, PEG, pegylated lipids and oligonucleotides have been developed to block the degradation of RNA in plasma and promote cellular uptake of nucleic acids.

Accordingly, as a result to extensive efforts to solve the problems described above and develop 5′-UTR with improved translation efficiency, the present inventors found that 5′-UTR with improved translation efficiency can be obtained by selecting artificial nucleic acid molecules that do not form secondary structures, contain less uridine, and do not contain sequences that lower stability from combinations of artificial nucleic acid molecules having a length of 30 bp and completed the present invention.

### SUMMARY OF THE INVENTION

It is one object of the present invention to provide a 5′-UTR polynucleotide with improved translation efficiency.

It is another object of the present invention to provide a synthetic nucleic acid molecule including the 5′-UTR polynucleotide with improved translation efficiency.

It is another object of the present invention to provide a vaccine composition including the synthetic nucleic acid molecule.

In accordance with one aspect of the present invention, the above and other objects can be accomplished by the provision of an isolated 5′-untranslated region (UTR) polynucleotide including a nucleotide sequence represented by a nucleic acid sequence of Formula (I) below:

AG[N₂₂]GCCACC Formula (I):

In accordance with another aspect of the present invention, provided is an isolated 5′-untranslated region (UTR) polynucleotide including a nucleotide sequence represented by a nucleic acid sequence of Formula (II) below:

AGGA[N₁₉]RGCCACC Formula (II):

wherein R represents A or G.

In accordance with another aspect of the present invention, provided is a synthetic nucleic acid molecule, in the order of 5′ to 3′, including a) a 5′-CAP structure, b) the 5′-UTR polynucleotide, c) at least one coding region, d) a 3′-untranslated region (3′-UTR), and e) 10 to 1,000 poly (A) tails or poly (A) tail-like sequences.

In accordance with another aspect of the present invention, provided is a vaccine composition including the synthetic nucleic acid molecule.

In accordance with another aspect of the present invention, provided is the use of the vaccine composition for the prevention of a disease.

In accordance with another aspect of the present invention, provided is a method for preventing a disease including administering the vaccine composition.

In accordance with another aspect of the present invention, provided is the use of the vaccine composition for the preparation of drugs for preventing a disease.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram illustrating a process for selecting a 5′-UTR candidate group according to an embodiment of the present invention;

FIG. 2 is a vector map of a vector formed to determine the in vitro transcription performance of mRNA containing 5′-UTR selected in an embodiment of the present invention;

FIG. 3 is a schematic diagram illustrating the structure of the mRNA containing the 5′-UTR selected in the embodiment of the present invention;

FIG. 4 illustrates the expression efficiency of the mRNA containing the 5′-UTR selected in the embodiment of the present invention in the HEK293T cell line (A) and (B), and in the HeLa cell line (C) and (D); and

FIG. 5 illustrates the expression efficiency of the mRNA containing the 5′-UTR selected in the embodiment of the present invention in the Huh7 cell line (A) and (B), and in the SNU423 cell line (C) and (D).

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

In the present invention, when the 5′-UTR with improved translation efficiency is determined by selection based on a certain logic from the entire combination rather than extraction from genes existing in nature, it exhibits superior performance to 5′-UTR existing in nature.

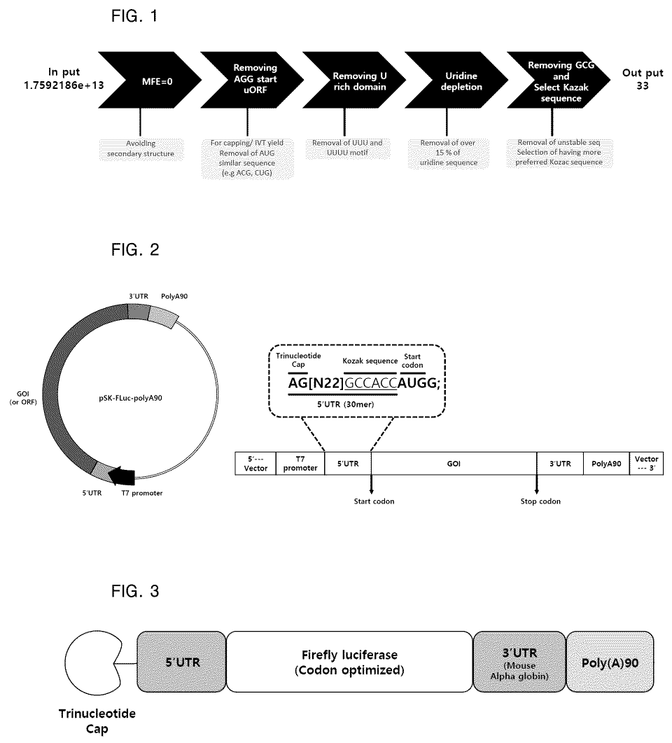

That is, in one embodiment of the present invention, a 5′-UTR polynucleotide selected by the following steps of removing a polynucleotide combination that may have a secondary structure from 30 polynucleotide combinations, as shown in FIG. 1, selecting a sequence to improve capping efficiency, removing UUU and UUUU motifs from the sequence, removing at least 15% uridine from the sequence, and removing a sequence with poor stability was found to exhibit improved translation efficiency (FIG. 1).

In one aspect, the present invention is directed to an isolated 5′-untranslated region (UTR) polynucleotide including a nucleotide sequence represented by a nucleic acid sequence of Formula (I) below:

AG[N₂₂]GCCACC Formula (I):

In another aspect, the present invention is directed to an isolated 5′-untranslated region (UTR) polynucleotide including a nucleotide sequence represented by a nucleic acid sequence of Formula (II) below:

AGGA[N₁₉]RGCCACC

wherein R represents A or G.

In the present invention, the 5′-UTR may be any one of the nucleotide sequences represented by SEQ ID NOs: 1 to 33.

TABLE 1

| Seq. ID | |
|---|---|
| No. | Sequence (5′ to 3′) |
| 1 | AGGAGAAGAGAGAAAAGUAAAAUAGCCACC |
| 2 | AGGAGUAAAAGUAAAAUAGUAAAAGCCACC |
| 3 | AGGAGAAAAGAAAGAGUAAAAUAGGCCACC |
| 4 | AGGAUUAAAAAUAGAAAGAAUAAGGCCACC |
| 5 | AGGACAACAAACAAAAACAAACUAGCCACC |
| 6 | AGGACAGAGCACUAGAACAAAACAGCCACC |
| 7 | AGGACCACUCUCAACCAAAACCUAGCCACC |
| 8 | AGGAGACACUAACACCCCGACCGAGCCACC |
| 9 | AGGACCGGAACACAAAAACUAGCAGCCACC |
| 10 | AGGAACUACACUAGCCACUAAACAGCCACC |
| 11 | AGGACAACUAGACAGAGACACCGAGCCACC |
| 12 | AGGACACAACACCCAGACCACCGAGCCACC |
| 13 | AGGACACAACUACCGAACUACUCAGCCACC |
| 14 | AGGAGAACUAGUACCCACCCGACAGCCACC |
| 15 | AGGAGAGAAAAAAACAACUACACAGCCACC |
| 16 | AGGAGAAACAACUAGGACCACAGAGCCACC |
| 17 | AGGACCUAAAACACCUCGACAGCAGCCACC |
| 18 | AGGAAAAAACUCACCUCUCAGACAGCCACC |
| 19 | AGGAGAGAAACUACACAGACUAGAGCCACC |
| 20 | AGGAACAACAGACUCACUCACUCAGCCACC |
| 21 | AGGAAAACUAAACAGAACCUAACAGCCACC |
| 22 | AGGAACAGAGAACACUAGAGAGCAGCCACC |
| 23 | AGGACCACACAGACCCACCUACUAGCCACC |
| 24 | AGGAAACCGACUAAAAACCCUCUAGCCACC |
| 25 | AGGAACAACACCAGCAGAACUCGAGCCACC |
| 26 | AGGAACAACAGUCGUAACCCUCUAGCCACC |
| 27 | AGGACCCUAACCAAACCCACUCUAGCCACC |
| 28 | AGGACACAACUCUACCCGACUACAGCCACC |
| 29 | AGGAACACAGUCAGCACCAACACAGCCACC |
| 30 | AGGACACUCAGACAACCCACACUAGCCACC |
| 31 | AGGACCUCAAAAAAAAACUCUCUAGCCACC |
| 32 | AGGACAACAGCUCACACCCCCCUAGCCACC |
| 33 | AGGACCCAGAACCCACCUAACAGAGCCACC |

As used herein, the term “UTR” refers to an untranslated region that is located upstream (5′) and/or downstream (3′) of a coding region of a nucleic acid molecule described herein and thus is typically present at the side of the coding region. Thus, the term “UTR” generally includes a 3′ untranslated region (“3′-UTR”) and a 5′-untranslated region (“5′-UTR”). A UTR may typically include or consist of a nucleic acid sequence that is not translated into a protein. Typically, the UTR includes a “regulatory element”.

As used herein, the term “regulatory element” refers to a nucleic acid sequence having the ability to affect gene regulatory activity, expression, in particular, transcription or translation of a transcribable nucleic acid sequence that is operably linked (via cis or trans). The term “regulatory element” includes promoters, enhancers, internal ribosome entry sites (IRES), introns, leaders, transcription termination signals such as polyadenylation signals and poly-U sequences and other expression regulatory elements. The regulatory element may act constitutively or in a time-and/or cell-specific manner. Optionally, the regulatory element exert may its function through interactions (e.g., recruitment and binding) of regulatory proteins capable of regulating (inducing, enhancing, reducing, abrogating or preventing) expression, particularly transcription of genes.

The UTR is preferably “operably linked”, i.e., located in a functional relationship, in a coding region in such a way that it controls (i.e., mediates or modulates, preferably enhances) the expression of the coding sequence.

As used herein, the term “5′-UTR” refers to a portion of a nucleic acid molecule, which is located 5′ (i.e., “upstream”) of an open reading frame and is not translated into a protein. In the context of the present invention, the 5′-UTR starts at the transcription start site and ends one nucleotide before the start codon of the open reading frame.

The 5′-UTR may contain an element that regulates gene expression, a so-called “regulatory element”. Such a regulatory element may be, for example, a ribosome-binding site. The 5′-UTR may be modified by post-transcriptional modification, for example, addition of 5′-CAP. Thus, the 5′-UTR preferably corresponds to a sequence of nucleic acid located between 5′-CAP and the start codon, in particular, a sequence of mature mRNA, and more specifically a sequence that extends from the nucleotide at the 3′ position of 5′-CAP, preferably, from the nucleotide immediately following the 3′-position of 5′-CAP to the nucleotide at the 5′ position of the start codon (transcription start site) of the protein coding sequence, preferably to the nucleotide immediately before the 5′ position of the start codon (transcription start site) of the protein coding sequence.

The nucleotide immediately following the 3′ position of the 5′-CAP of the mature mRNA typically corresponds to the transcription initiation site. The length of a 5′ UTR is generally less than 500, 400, 300, 250 or 200 nucleotides. In some embodiments, the length of 5′ UTR is 10, 20, 30, 40 or more, preferably 10 or 50 or less nucleotides.

In another aspect, the present invention is directed to a synthetic nucleic acid molecule, in the order of 5′ to 3′, including a) a 5′-CAP structure, b) the 5′-UTR polynucleotide, c) at least one coding region, d) a 3′-untranslated region (3′-UTR), and e) 10 to 1,000 poly (A) tails or poly (A) tail-like sequences.

5′-CAP of native mRNA is involved in nuclear export, increases mRNA stability, and binds to mRNA cap-binding protein (CBP), which results in mRNA stability at the cellular and translational stage through association of the poly (A)-binding protein with CBP to form a mature cyclic mRNA species. The cap further aids in the removal of the 5′ proximal intron during mRNA splicing.

5′-CAP according to the present invention is typically modified nucleotide (CAP analog), in particular a guanine nucleotide added to the 5′ end of an mRNA molecule. Preferably, 5′-CAP is added using a 5′-5′-triphosphate linkage (also called “m7GpppN”). Further, examples of 5′-CAP structures include glyceryl, inverted deoxy abasic residues (moieties), 4′,5′-methylene nucleotides, 1-(beta-D-erythrofuranosyl) nucleotides, 4′-thio nucleotide, carbocyclic nucleotides, 1,5-anhydrohexitol nucleotides, L-nucleotides, alpha-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′,3′-inverted nucleotide moieties, 3′,3′-inverted abasic moieties, 3′,2′-inverted nucleotide moieties, 3′,2′-inverted abasic moieties, 1,4-butanediol phosphates, 3′-phosphoramidates, hexylphosphates, aminohexyl phosphates, 3′-phosphate, 3′-phosphorothioates, phosphorodithioates, or bridging or non-bridging methylphosphonate moieties.

These modified 5′-CAP structures may be used to modify the mRNA sequence of the synthetic nucleic acid molecule of the present invention.

Additional modified 5′-CAP structures that may be used in the present invention include CAP1 (additional methylation of the ribose of the adjacent nucleotide of m7GpppN), CAP2 (additional methylation of the ribose of the second nucleotide downstream of m7GpppN), CAP3 (additional methylation of the ribose of the third nucleotide downstream of m7GpppN), CAP4 (additional methylation of the ribose of the fourth nucleotide downstream of m7GpppN), ARCA (anti-reverse CAP analogue), modified ARCA (e.g., phosphothioate-modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In the present invention, the 5′-CAP structure may be formed by in vitro transcription (co-transcriptional capping) using chemical RNA synthesis or cCAP analog, or the CAP structure may be formed in vitro using a capping enzyme (e.g., a commercially available capping kit).

In the present invention, the CAP analog refers to a non-polymerizable di-nucleotide having a CAP function to promote translation or localization and/or prevent degradation of the RNA molecule when introduced at the 5′ end of an RNA molecule. Non-polymerizable means that since the CAP analog does not have 5′ triphosphate, it is bound only at the 5′ end and thus cannot be extended in the 3′ direction by a template-dependent RNA polymerase.

The CAP analogs include a chemical structure selected from the group consisting of: m7GpppA, m7GpppAmpG, unmethylated CAP analogs; dimethylated CAP analogs, trimethylated CAP analogs (e.g., m2,2,7GpppA), dimethylated symmetric CAP analogs (e.g., m7Gpppm7A), or anti-inverted CAP analogs (e.g., ARCA; m7,2 ′OmeGpppA, m7,2′dGpppA, m7,3′OmeGpppA, m7,3′dGpppA and tetraphosphate derivatives thereof), but are not limited thereto.

Additional CAP analogs are disclosed in the prior art (U.S. Pat. No. 7,074,596, WO 2008/016473, WO 2008/157688, WO 2009/149253, WO 2011/015347, and WO 2013/059475).

In the present invention, the 5′-CAP structure may be selected from the group consisting of m7GpppAmpG, m7,3′OmeApppG and m7GpppA, but is not limited thereto.

In the present invention, the coding region may encode at least one protein selected from the group consisting of antigenic proteins, allergenic proteins, therapeutic proteins, and fragments, mutants or derivatives of the protein, but is not limited thereto.

In the present invention, the antigenic protein may include at least one selected from the group consisting of tumor antigens, pathogenic antigens, autoantigens, alloantigens and allergens, but is not limited thereto.

As used herein, the term “tumor antigen” refers to an antigenic (poly-)peptide or protein derived from or related to a (preferably malignant) tumor or cancer disease. As used herein, the terms “cancer” and “tumor”, which are used interchangeably, refer to a neoplasm of cells that invade surrounding tissues and metastasize to distant body parts, characterized in that the cells are uncontrolled and generally rapidly proliferate. The term includes benign and malignant neoplasms. Malignant of cancer are tumors typically characterized by anaplasia, invasiveness, and metastasis; and benign malignancies usually do not have these characteristics. The terms “cancer” and “tumor” refer particularly to neoplasms characterized by tumor growth, as well as cancers of the blood and lymphatic systems. The “tumor antigen” is usually derived from tumor/cancer cells, preferably mammalian tumor/cancer cells, and may be located inside or on the surface of tumor cells and tumors, for example, systemic or solid tumors derived from mammals, preferably humans. The term “tumor antigen” generally includes tumor-specific antigens (TSA) and tumor-associated-antigens (TAA). The TSA is usually derived from tumor-specific mutations and is specifically expressed by tumor cells. The more common TAAs are usually presented by tumors and “normal” (healthy and non-tumor) cells.

As used herein, the term “tumor antigen” may be a protein or nucleic acid sequence associated with a tumor, wherein each nucleic acid sequence encodes another peptide or protein, and the at least one nucleic acid sequence may encode 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, A T-4, ARTC1/m, B7H4, BAGE-1, BCL-2, bcr/abl, beta-catenin/m, BING-4, BRCA1/m, BRCA2/m, CA 1 5-3/CA 27-29, CA 19-9, CA72-4, CA125, calreticulin, CAMEL, CASP-8/m, cathepsin B, cathepsin L,

CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERVK-MEL, HLA-A*0201-R1 7I, HLA-A1 1/m, HLA-A2/m, HNE, homeobox NKX3.1, HOM-TES-14/SCP-1,

HOM-TES-85, HPV-E6, HPV-E7, HSP70-2M, HST-2, hTERT, iCE, IGF-1 R, IL-13Ra2, IL-2R, IL-5, immature laminin receptors, kallikrein-2, kallikrein-4, 167, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGED2, MAGE-D4, MAGE-E1, MAGE-E2, MAGE-F1, MAGE-HI, MAGEL2, mammaglobin A, MART-1/Melan-A, MART-2, MART-2/m, matrix protein 22, MC1 R, M-CSF, ME 1/m, mesothelin, MG50/PXDN, MMP1-1, MN/CA IX-antigen, MRP-3, MUC-1, MUC-2, MUM-1/m, MUM-2/m, MUM-3/m, myosin class 1/m, NA88-A, N-acetylglucosaminyltransferase-V, Neo-PAP, Neo-PAP/m, NFYC/m, NGEP, NMP22, NPM/ALK, N-Ras/m, NSE, NY-ESO-1, NY-ESOB, OA1, OFA-iLRP, OGT, OGT/m, OS-9, OS-9/m, osteocalcin, osteopontin, pi 5, p190 minor bcr-abl, p53, p53/m, PAGE-4, PAI-1, PAI-2, PAP, PART-1, PATE, PDEF, Pim-1-kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGF-beta, TGF-beta RII, TGM-4, TPI/m, TRAG-3, TRG, TRP-1, TRP-2/6b, TRP/INT2, TRP-p8, tyrosinase, UPA, VEGFR1, VEGFR-2/FLK-1, WT1, and the immunoglobulin genotype of lymphocytes or the T cell receptor genotype of lymphocytes, or a homologue, fragment, variant or derivative of the tumor antigen.

In the present invention, the tumor antigen is selected from the group consisting of NYESO-1, HER-2/neu, MAGE-1, tyrosinase, MUC1, CEA, Mam-A, hTERT, Syalyl-Tn, WT1, alpha-fetoprotein, CA-125, gp-100, p53, Ras, Src, EGFRVIII, PSMA, GD2, Bcr-abl, survivin, PSA, EphA2, PAP, AFP, EpCAM, ALK, Mesothelin, PSCA, MART-1, Melan-A, SCP-1, SPAG9, AKAP4 and OY-TES-1, but is not limited thereto.

In the present invention, the pathogenic antigen may be selected from the group consisting of bacterial, viral, fungal and protist antigens.

In the present invention, the pathogenic antigen may be derived from influenza virus, respiratory syncytial virus

(RSV), coronavirus, herpes simplex virus (HSV), human papillomavirus (HPV), human immunodeficiency virus (HIV), plasmodium, Staphylococcus aureus, dengue virus, trachoma chlamydia, cytomegalovirus (CMV), hepatitis B virus (HBV), mycobacterium tuberculosis, rabies virus, and yellow fever virus, or isoforms, homologues, fragments, variants or derivatives of such proteins.

In the present invention, the viral antigen may be a corona virus, but is not limited thereto.

In the present invention, the coronavirus is human coronavirus 229E (HCOV-229E), human coronavirus OC43 (HCoV-OC43), severe acute respiratory syndrome coronavirus (SARS-CoV), human coronavirus NL63 (HCOV-NL63, New Haven coronavirus), human coronavirus HKU1, middle east respiratory syndrome coronavirus (MERS-COV) or severe acute respiratory syndrome coronavirus 2 (SARS-Cov-2).

In the present invention, the 3′-UTR is selected from the group consisting of α-globin 3′-UTR, CYBA 3′-UTR, albumin 3′-UTR, growth hormone (GH) 3′-UTR, VEEV 3′-UTR; hepatitis B virus (HBV) 3′-UTR, α-globin 3′-UTR, DEN 3′-UTR, Barley Yellow Dwarf Virus-PAV (BYDV-PAV) 3′-UTR, elongation factor 1 α1 (EEF1A1) 3′-UTR, manganese peroxide dismutase (MnSOD) 3′-UTR, β subunit (β-mRNA) 3′-UTR of mitochondrial H(+)-ATP synthase, GLUT1 3′-UTR, MEF2A 3′-UTR, β-F1-ATPase 3′-UTR, and functional fragments thereof and combinations thereof, but is not limited thereto.

As used herein, the term “3′-UTR” typically refers to a moiety of mRNA located between the protein coding region (i.e., open reading frame, coding region) of the mRNA and the poly(A) sequence. The 3′-UTR of mRNA is not translated into an amino acid sequence. The 3′-UTR sequence is usually encoded by the gene that is transcribed into each mRNA during the gene expression process. This genomic sequence is first transcribed into immature mRNA including selective introns. The immature mRNA is then further processed into mature mRNA in the maturation process. This maturation process includes steps such as 5′-capping, splicing of immature mRNA to excise selective introns, and modifications of the 3′ terminus such as polyadenylation of the 3′ terminus of immature mRNA and selective endo-or exonuclease cleavage.

In the present invention, the 3′-UTR corresponds to the sequence of mature mRNA that is present from the nucleotide at the 3′ position of the stop codon of the protein coding region to the nucleotide at the 5′ position of the poly (A) sequence, preferably at the 5′ position immediately next to the poly(A) sequence. The term “corresponding” means that the 3′-UTR sequence may be an RNA sequence, such as an mRNA sequence used to define a 3′-UTR sequence, or a DNA sequence corresponding to such an RNA sequence.

In the present invention, the synthetic nucleic acid molecule further includes a poly (A) tail or a poly (A) tail-like sequence. In further embodiments, terminal groups on the poly-A tail may be incorporated for stabilization. In another embodiment, the poly-A tail includes a des-3′ hydroxyl tail.

During RNA processing, long chains of adenine nucleotides (poly-A tails) may be added to polynucleotides, such as mRNA molecules, in order to increase stability. Immediately after transcription, the 3′ end of the transcript may be cleaved to liberate the 3′ hydroxyl. Then, poly-A polymerase then adds an adenine nucleotide chain to the RNA. A process called “polyadenylation”, for example, adds a polyA tail that may be approximately 80 to approximately 250 residues in length (about 80, 90, 100, 110, 120, 130, 140, 150, 160, 170, 180, 190, 200, 210, 220, 230, 240 or 250 residues in length). The polyA tail may also be added after the construct has exited the nucleus.

According to the present invention, the terminal groups on the poly A tail may be incorporated for stabilization. The polynucleotides of the present invention may include a des-3′ hydroxyl tail. The polynucleotides may also include moieties structural or 2′-Omethyl modifications, as suggested by Junjie Li et al. (Current Biology, Vol. 15, 1501-1507 Aug. 23, 2005, the contents of which are incorporated herein by reference in their entirety).

The unique poly-A tail length provides predetermined advantages of the polynucleotides of the present invention. In general, the poly-A tail, if present, is greater than 30 nucleotides in length. In other embodiments, the poly-A tail is greater than 35 nucleotides in length (e.g., at least about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500 and 3,000 nucleotides).

In some embodiments, the polynucleotide or region thereof includes from about 30 and about 3,000 nucleotides (for example, 30 to 50, 30 to 100, 30 to 250, 30 to 500, 30 to 750, 30 to 1,000, 30 to 1,500, 30 to 2,000, 30 to 2,500, 50 to 100, 50 to 250, 50 to 500, 50 to 750, 50 to 1,000, 50 to 1,500, 50 to 2,000, 50 to 2,500, 50 to 3,000, 100 to 500, 100 to 750, 100 to 1,000, 100 to 1,500, 100 to 2,000, 100 to 2,500, 100 to 3,000, 500 to 750, 500 to 1,000, 500 to 1,500, 500 to 2,000, 500 to 2,500, 500 to 3,000, 1,000 to 1,500, 1,000 to 2,000, 1,000 to 2,500, 1,000 to 3,000, 1,500 to 2,000, 1,500 to 2,500, 1,500 to 3,000, 2,000 to 3,000, 2,000 to 2,500, and 2,500 to 3,000).

In some embodiments, the poly-A tail is designed for the length of the entire polynucleotide or the length of a specific region of the polynucleotide. This design may be based on the length of the coding region, a specific feature or the length of specific region, or on the length of the ultimate product expressed from the polynucleotide.

In this regard, the poly-A tail may be 10, 20, 30, 40, 50, 60, 70, 80, 90 or 100% greater in length than the polynucleotide or feature thereof. The poly-A tail may also be designed as a fraction of the polynucleotide to which it belongs. In this regard, the poly-A tail may be at least 10, 20, 30, 40, 50, 60, 70, 80, or 90% of the total length of the construct, the construct region or the value obtained by subtracting the poly-A tail from the total length of the construct. Additionally, engineered binding sites and conjugation of polynucleotides to poly-A binding proteins can enhance expression.

Additionally, multiple distinct polynucleotides may be linked together via PABP (poly-A binding protein) through the 3′-end using modified nucleotides at the 3′-end of the poly-A tail. Transfection experiments may be performed in appropriate cell lines and protein production may be assayed by ELISA at 12 hours, 24 hours, 48 hours, 72 hours and 7 days after transfection.

In some embodiments, the polynucleotides of the present invention are designed to include polyA-G quartet regions. The G-quartet is a cyclic hydrogen-bonded structure of four guanine nucleotides that may be formed by G-rich sequences in both DNA and RNA. In this experiment, the G-quartet is incorporated at the end of the poly-A tail. The resulting polynucleotides are assayed for other parameters including stability, protein production and half-life at various time points. It was found that the polyA-G quartet resulted in protein production from mRNA equivalent to at least 75% that can be found using a poly-A tail of only 120 nucleotides.

In the present invention, the poly (A) tail-like sequence may be used without limitation as long as it is a nucleic acid sequence capable of performing the function of the poly (A) tail, preferably at least one nucleotide other than adenine selected from the group consisting of uracil (U), cytosine (C) and guanine (G) inserted between a plurality of adenines or at the end of the poly (A) tail, but is not limited thereto.

In the present invention, the synthetic nucleic acid molecule may be RNA.

In the present invention, the RNA may be selected from the group consisting of mRNA, viral RNA, self-replicating RNA and replicon RNA, but is not limited thereto.

In the present invention, the synthetic nucleic acid molecule may include at least one backbone-modified, sugar-modified or base-modified nucleic acid, but is not limited thereto.

### Sugar Modification:

Modified nucleosides and nucleotides that may be incorporated into a modified mRNA compound including an mRNA sequence as described herein may be modified at the sugar moiety. For example, the 2′ hydroxyl group (OH) may be modified or replaced with a number of different “oxy” or “deoxy” substituents. Examples of the “oxy”-2″ hydroxyl modification include: alkoxy or allyloxy (—OR, e.g., R═H, alkyl, cycloalkyl, aryl, aralkyl, heteroaryl, or sugar); polyethylene glycol (PEG), —O(CH₂CH₂O)ₙCH₂CH₂OR; “locked” nucleic acids (LNAs) in which the 2′ hydroxyl is linked to the 4′ carbon of the same ribose sugar, for example, by methylene bridge; and an amino group (—O-amino, wherein the amino group, for example, NRR is alkylamino, dialkylamino, heterocyclyl, arylamino, diarylamino, heteroarylamino, diheteroaryl amino, ethylenediamine, or polyamino) or aminoalkoxy, but are not limited thereto.

The “deoxy” modification includes hydrogen, amino (e.g., NH₂; alkylamino, dialkylamino, heterocyclyl, arylamino, diarylamino, heteroarylamino, diheteroarylamino, or amino acid), or the amino group may be attached to the sugar through a linker, wherein the linker includes one or more of the atoms C, N, and O.

The sugar group may also include at least one carbon having a stereochemical configuration opposite to the corresponding carbon in the ribose. Thus, the modified mRNA may include nucleotides containing arabinose, for example, a sugar.

### Backbone Modification:

The phosphate backbone may be further modified at modified nucleosides and nucleotides, which may be incorporated into modified mRNA compounds including mRNA sequences as described herein. The phosphate of the backbone may be modified by replacing at least one oxygen atom with another substituent. In addition, the modified nucleosides and nucleotides may include complete replacement of an unmodified phosphate moiety with modified phosphate as described herein. Examples of the modified phosphate include, but are not limited to, phosphorothioates, phosphoroselenates, boranophosphates, boranophosphate esters, hydrogen phosphonates, phosphoroamidates, alkyl or aryl phosphonates, and phosphotriesters. The phosphorodithioates have both unlinked oxygens replaced by sulfur.

Phosphate linkers may also be modified by substitution of the linking oxygen with nitrogen (bridged phosphoroamidate), sulfur (bridged phosphorothioate) and carbon (bridged methylene-phosphonate).

### Base Modification:

Modified nucleosides and nucleotides that may be incorporated into a modified mRNA compound including an mRNA sequence as described herein may be further modified at the nucleobase moiety. Examples of nucleobases found in mRNA include, but are not limited to, adenine, guanine, cytosine, and uracil. For example, the nucleosides and nucleotides described herein may be chemically modified at the major groove face. In some embodiments, the chemical modification of major groove may include amino, thiol, alkyl, or halo groups. In a preferred embodiment of the present invention, the nucleotide analogues/modifications are preferably determined by base modifications selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurine riboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, puromycin-5′-triphosphate, and xanthosine-5′-triphosphate.

Preferred are base-modified nucleotides selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, the modified nucleoside includes pyridin-4-one ribonucleoside, 5-aza-uridine, 2-thio-5-aza-uridine, pseudouridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 2′-O-methyl-uridine, 5-methyl-uridine, 5-propynyl-uridine, 1-propynyl-pseudouridine, 5-taurinomethyluridine, 2-thio-uridine, 5-methoxy-uridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, the modified nucleoside includes 5-aza-cytidine, pseudoisocytidine, 3-methyl-cytidine, 5-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, the modified nucleoside includes 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentyladenosine, N6-(cis-hydroxyisopentyl)adenosine, 2-methylthio-N6-(cis-hydroxyisopentenyl)adenosine, N6-glycinylcarbamoyladenosine, N6-threonylcarbamoyladenosine, 2-methylthio-N6-threonylcarbamoyladenosine, N6,N6-dimethyladenosine, 7-methyladenine, 2-methylthio-adenine, and 2-methoxy-adenine.

In other embodiments, the modified nucleoside includes inosine, 1-methyl-inosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide may be modified at the major groove face and may include replacement of the hydrogen at the C-5 of uracil with a methyl group or a halo group. In certain embodiments, the modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In a further embodiment, the modified mRNA may include nucleoside modification selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, and 7-deaza-adenosine.

In another aspect, the present invention is directed to a vaccine composition including the synthetic nucleic acid molecule.

The term “vaccine” is considered a prophylactic or therapeutic substance that typically provides at least one antigen, preferably an antigenic peptide or protein. “Provides at least one antigen” means that, for example, the vaccine includes an antigen or that the vaccine includes, for example, a molecule encoding the antigen. Thus, the vaccine of the present invention may be derived from, for example, a tumor antigen, bacterial, viral, fungal or protozoan antigen, autoantigen, allergen, or allogeneic antigen, and is preferably particularly envisaged to include at least one synthetic nucleic acid (RNA) molecule encoding at least one antigenic (poly-)peptide or protein as defined herein, which induces an immune response against each antigen, when expressed in and presented to the immune system. However, synthetic nucleic acid (RNA) molecules encoding non-antigenic (poly-)peptides or proteins of interest may also be used in the vaccine of the present invention.

In the present invention, the synthetic nucleic acid molecule may be complexed with one or more lipids to form lipid nanoparticles or liposomes.

The synthetic nucleic acid (RNA) molecule in the present invention is complexed or associated with, i.e., one or more (poly-)cationic compounds, preferably (poly-)cationic polymers, (poly-)cationic peptides or proteins, for example protamine, (poly-)cationic polysaccharide and/or (poly-)cationic lipid. In this context, the term “complexed” or “associated” means a combination of at least one synthetic nucleic acid (RNA) molecule with the at least one compound that is intrinsically stable as a larger complex or assembly without a covalent bond with the same.

### Lipid

In a preferred embodiment, the synthetic nucleic acid (RNA) molecules of the present invention are complexed or associated with lipids (especially cationic and/or neutral lipids) to form one or more lipid nanoparticles or liposomes. Thus, in some embodiments, the synthetic nucleic acid (RNA) molecules of the present invention may be provided in the form of lipid-based formulations, in particular in the form of liposomes and/or lipid nanoparticles including the synthetic nucleic acid (RNA) molecules.

### Lipid Nanoparticles

In some preferred embodiments, synthetic nucleic acid (RNA) molecules of the present invention are complexed or associated with lipids (especially cationic and/or neutral lipids) to form one or more lipid nanoparticles.

Preferably, the lipid nanoparticles (LNP) may include the following components: (a) at least one synthetic nucleic acid molecule (RNA) of the present invention; (b) a cationic lipid; (c) an aggregation reducing agent (e.g., for example polyethylene glycol (PEG) lipid or PEG-modified lipid); (d) optionally a non-cationic lipid (e.g., neutral lipid); and (e) optionally, sterol.

In some embodiments, the LNP may include, in addition to at least one synthetic nucleic acid molecule (RNA) of the present invention, (i) at least one cationic lipid, (ii) a neutral lipid, and (iii) sterol such as cholesterol, and PEG-lipid, wherein the cationic lipid is present in an amount of about 20 to 60%, the neutral lipid is present in an amount of 5 to 25%, the sterol is present in an amount of 25 to 55% and the PEG-lipid is present in an amount of 0.5 to 15%.

In some embodiments, the synthetic nucleic acid molecule (RNA) of the present invention may be formulated into aminoalcohol lipidoid. The aminoalcohol lipidoid that may be used in the present invention may be prepared by the method described in U.S. Pat. No. 8,450,298, which is incorporated herein by reference in its entirety.

### Liposome

In some embodiments, the synthetic nucleic acid (RNA) molecules of the present invention are formulated into liposomes. The cationic lipid-based liposomes may form complexes with negatively charged nucleic acids (e.g., RNA) through electrostatic interactions, resulting in formation of complexes that offer possibilities of biocompatibility, low toxicity, and mass productivity required for in vivo clinical applications. Liposomes may be fused with the plasma membrane for absorption; first, the liposomes are processed through the phagocytosis pathway inside the cells and the nucleic acid is then released into the cytoplasm from the endosome/carrier. Liposomes have long been recognized as drug delivery vehicles due to excellent biocompatibility in that liposomes are basically analogs of biological membranes and may be prepared from both natural and synthetic phospholipids.

Liposomes are typically formed of a lipid bilayer, which may contain cationic, anionic or neutral (phospho) lipids and cholesterol, surrounding an aqueous core. Both the lipid bilayer and the aqueous core may contain hydrophobic or hydrophilic compounds. Liposomes may have one or more lipid membranes. Liposomes may be a single layer, called “unilamellar liposome”, or multiple layers, called “multilamellar liposomes”.

Liposome properties and behaviors in vivo may be modified by coating with a hydrophilic polymer, such as, by adding polyethylene glycol (PEG) to the liposome surface to provide steric stability. In addition, liposomes may be used for specific targeting by adhering ligands (e.g., antibodies, peptides and carbohydrates) to the surface thereof or to the end of the adhered PEG chain.

Liposomes typically exist as spherical vesicles and can range in size from 20 nm to several microns. Liposomes may have different diameters such as multilamellar vesicles (MLV), which may be several hundred nanometers in diameter and may contain a series of concentric bilayers separated by narrow aqueous compartments, small unicellular vesicles (SUV) smaller than 50 nm in diameter, and large unilamellar vesicles (LUVs), which may be between 50 and 500 nm in diameter, but are not limited thereto. Liposome designs may include, but are not limited to, opsonins or ligands to improve adhesion of liposomes to unhealthy tissue or to activate events such as, but not limited to, endocytosis. Liposomes may have a low or high pH to enhance delivery of the pharmaceutical formulation.

In the present invention, the vaccine composition may further include one or more adjuvants or active agents.

In the broadest sense, the term “adjuvant” or “adjuvant component” is typically a pharmacological and/or immunological agent capable of modifying, e.g., enhancing, the effectiveness of another active agent, e.g., therapeutic agent or vaccine. In this context, an “adjuvant” may be considered as any compound suitable to assist in the administration and delivery of the vaccine composition of the present invention. Specifically, the adjuvant preferably enhances the immune-stimulating property of the added vaccine. In addition, the adjuvant is not bound thereto and initiates or increases the immune response of the innate immune system, i.e., a non-specific immune response.

The “adjuvant” typically does not induce an adaptive immune response. To date, the “adjuvant” is not qualified as an antigen. That is, when administered, the vaccine of the invention typically initiates an adaptive immune response due to the antigenic peptide or protein encoded by at least one coding sequence of the synthetic nucleic acid (RNA) molecule contained in the vaccine.

Suitable adjuvants are known to those skilled in the art and may be selected from any adjuvant suitable in the present case, i.e., assisting in the induction of an immune response in a mammal, and may include: TDM, MDP, muramyl dipeptide, Pluronic, alum solution, aluminum hydroxide, ADJUMERTM (polyphosphazene); aluminum phosphate gel; glucans from algae; algamulin; aluminum hydroxide gel (alum); high protein-adsorption aluminum hydroxide gel; low-viscosity aluminum hydroxide gel; AF or SPT (emulsion of squalane (5%), Tween 80 (0.2%), Pluronic L121 (1.25%), phosphate-buffered saline, pH 7.4); or AVRIDINE™ (propanediamine), but is not limited thereto.

In another aspect, the present invention is directed to the use of the vaccine composition for the prevention of a disease.

As used herein, the term “disease” refers to an abnormality occurring from tumor antigens, bacteria, viruses, fungal or protozoan antigens, autoantigens, allergens, or allogeneic antigens, and may be cancer, tumor, an autoimmune disease, an inflammatory disease, a viral infection, a bacterial infection, a fungal infection, a protozoan infection, or the like, but is not limited thereto.

As used herein, the term “prevention” means any action that suppresses a disease or delays the progression thereof by administration of the vaccine composition of the present invention.

In another aspect, the present invention is directed to a method for preventing a disease including administering the vaccine composition.

In another aspect, the present invention is directed to the use of the vaccine composition for the preparation of drugs for preventing a disease.

### EXAMPLE

Hereinafter, the present invention will be described in more detail with reference to the following examples. However, it will be obvious to those skilled in the art that the following examples are provided only for illustration of the present invention and should not be construed as limiting the scope of the present invention.

### Example 1. 5′-UTR Selection

All possible combinations of 5′-UTR polynucleotides with a size of 30 bp were constructed, sequences with a mean free energy of 0 that does not form a specific structure such as a secondary structure were selected, sequences that start with an AGG sequence were selected to improve capping and transcription, and AUG-like sequences were further removed from some sequences. Since immune sensing of TLR7/8 by uridine-rich element reduces protein translation efficiency, the sequence containing the UUU and UUUU motifs was removed and uridine depletion to remove 15% of uridine from the entire sequence was performed in order to increase protein translation efficiency. To improve mRNA stability, 5′-UTR having a CGC sequence was removed to select a total of 33 5′-UTR sequences (FIG. 1).

### Example 2. Preparation of Nucleic Acid for Synthesis of 5′-UTR-Containing mRNA

### 2-1. Linearization for Template DNA Preparation

Linearization was performed using a restriction enzyme that cuts the downstream of the poly A site in the plasmid. The linearized DNA was isolated using AMICON.

Then, whether or not the plasmid DNA was cut was identified on a 1% agarose gel.

### 2-2. In Vitro Transcription

In vitro transcription is the process of synthesizing mRNA.

The prepared template DNA was reacted with T7 RNA polymerase, buffer, NTP (including natural and chemically modified NTP) and other necessary elements of IVT at 37° C. for 4 hours (hr) as shown in Table 2 (HiScribe™ T7 Quick High Yield RNA Synthesis Kit, NEB E2050S, US).

TABLE 2

| Components | Volume | |
|---|---|---|
| NTP mix (Final 10 mM) | 2 | μL |
| RNase Inhibitor | 0.5 | μL |
| Pyrophosphatase, inorganic | 0.4 | μL |
| (Yeast) | | |
| 100 mM Clean Cap AG | 2 | μL |
| Template DNA | 1 | μg |
| 10X T7 Polymerase reaction | 2 | μL |
| Buffer | | |
| T7 RNA polymerase mix | 2 | μL |
| Nuclease free water | Up to 20 μL | |
| Total | 20 | μL |

When the reaction was complete, 1 ug of DNA was treated with 1U of DNase I, followed by reaction at 37° C. for 15 to 30 minutes (min) to remove template DNA. After the reaction was completed, the IVT product was purified by the method suggested by the manufacturer of Invitrogen's MEGAclear™ kit (Austin, Tex.). Then, the purified mRNA was quantified by Nanodrop or UV/Vis absorption and the band pattern was identified on an agarose gel.

### 2-3. DsRNA Purification

The double-stranded RNA (dsRNA) produced during the IVT reaction was removed using cellulose.

Cellulose prewash:

0.2 g of cellulose was released in 1 ml of buffer A in a 50 ml tube and then a bench top-scale cellulose column was prepared. The compositions of buffers A and B are shown in Table 3 below.

TABLE 3

| | Conc. | Stock conc. | Need vol. |
|---|---|---|---|
| 1X Buffer A | (mM) | (mM) | (mL) |
| HEPES | 10 | 1000 | 0.4 |
| EDTA | 0.1 | 5 | 0.8 |
| NaCl | 125 | 5000 | 1 |
| Ethanol | 16% | 100% | 6.4 |
| NaOH | — | 25000 | 0.02 |
| DW | | | 31.38 |
| Total | | | 40 |
| | | Stock conc. | Need vol. |
| 2X Buffer A | Conc. (mM) | (mM) | (mL) |
| HEPES | 20 | 1000 | 0.8 |
| EDTA | 0.2 | 5 | 1.6 |
| NaCl | 250 | 5000 | 2 |
| Ethanol | 32% | 100% | 12.8 |
| NaOH | — | 25000 | 0.035 |
| DW | | | 22.765 |
| Total | | | 40 |

IVT mRNA loading:

IVT mRNA dissolved in 500 μl buffer A was charged into the column prepared above.

Reaction was induced at room temperature for 30 minutes using a rotator to bind cellulose to dsRNA.

The result was centrifuged at 14,000 g for 1 minute, and flow-through was recovered and was transferred to a new column.

Reaction was performed again at room temperature for 30 minutes, centrifugation was performed under the same conditions and the flow through is recovered.

mRNA precipitation:

The pellet was recovered by isopropanol precipitation and dissolved in nuclease free water.

### Example 3. Determination of Purity of Prepared mRNA

### 3-1. IP-RP UPLC

In order to determine the purity of the mRNA, the mRNA was analyzed using a reversed phase chromatography column such as C18 or C8.

Water-based buffer and ACN-based buffer containing alkyl ammonium acetate-based ion-pairing additives (ex. TBAA, TPAA, DMBAA, HAA, etc.) containing TEAA were used as mobile phases for chromatography analysis. Organic solvents such as acetonitrile and methanol were used to wash and store the column. Buffer having a high water content was used as weak wash buffer, and buffer containing 10-50% organic solvent were used as strong wash buffer and seal wash buffer.

TEAA in water-based buffer was connected to Pump A, TEAA in ACN-based buffer was connected to Pump B, and an initial priming process was performed. The column was connected to the device, at least 5 CV was applied to the column under the initial gradient condition and the equilibrium was maintained. Whether or not Δpsi was stabilized during the equilibrium was determined.

mRNA was injected and then analyzed under the gradient in which an organic solvent ratio increases as compared to an initial stage.

After analysis, organic solvents such as acetonitrile and methanol were used for column washing and storage, and at least 10 CV was applied.

### 3-2. Dot Blot

Dot blot was performed to qualitatively determine double-stranded RNA (dsRNA) as an impurity.

The J2 antibody was bound to the dsRNA and was thus used as a probe to determine the presence or absence of the dsRNA.

The IVT mRNA to be analyzed was dropped in a volume of 2 μL with a concentration of 100-200 ng/μL into a positively charged nylon membrane (Merck/11417240001) and completely dried at room temperature for at least 1 hour.

The sample was crosslinked one cycle into the membrane at 1,250 uJ/CM2 using a UV cross linker, and reacted with 4% skim milk (in 1× TBST) blocking solution on a shaker at room temperature for 1 hour.

A J2 primary antibody was diluted 1:5000 in blocking solution and reacted overnight at 4° C. using a shaker.

The result was washed 3 times with 1× TBST solution using a shaker at room temperature for 10 minutes, and the goat anti-mouse (IgG) secondary antibody conjugated with horseradish peroxidase (HRP) was diluted 1:5000 in blocking solution and reacted using a shaker at room temperature for 1 hour.

Then, the result was washed 3 times with 1× TBST solution using a shaker at room temperature for 10 minutes.

ECL reagent 1 and 2 solutions were mixed at 1:1 (v/v), the membrane was immersed in the mixture and was exposed to light for 10 seconds, and the band was visualized using Chemi Doc.

### Example 4. Determination of Translation Efficiency of Prepared mRNA

The evaluation of translation efficiency of the prepared mRNA was identified by luciferase activity.

Human embryonic kidney HEK293T (human kidney embryonic cell line, ATCC CRL-3216) and Hela (human cervix epithelial cell, CCL2) cells were cultured in DMEM/Dulbecco's modified Eagles medium supplemented with 10% fetal bovine serum (FBS) and 1% penicillin/streptomycin, and Huh7 (human liver cancer cell line, Korea cell line bank, 60104) and SNU423 (human liver cancer cell line, Korea cell line bank, 00423) cells were cultured in RPMI-1640 (Gibco) media supplemented with 10% fetal bovine serum (FBS) and 1% penicillin/streptomycin.

250 ng of mRNA was transfected in a 12-well plate with lipofectamine 3000 (Invitrogen, Carlsbad, Calif).

The controls used herein are as follows:

1. Human alpha globin A (WO 2020/198337-derived sequence modification):

(SEQ ID NO: 34)
AGTCTTCTGGTCCCCACAGACTCAGAGAGAACCCACC

2. Cytochrome b-245 alpha chain CYBA (WO 2020/198337):

(SEQ ID NO: 35)
AGTGCGCGCCTAGCAGTGTCCCAGCCGGGTTCGTGTCGCC

3. Ref UTR no. 1 (US2020/0208145):

(SEQ ID NO: 36)
AGGAAATAAGAGAGAAAAGAAGAGTAAGAAGAAATATAAGAGCCACC

4. Ref UTR no. 2 (US2020/0208145):

(SEQ ID NO: 37)
GGGAAATAAGAGAGAAAAGAAGAGTAAGAAGAAATATAAGACCCCGG
CGCCGCCACC

20 μL of bright-glo luciferase assay reagent was added to the transfected 96-well plate, and then the 96-well plate was allowed to stand for 1 minute and then shaken at room temperature for 10 minutes.

The integration time was set to 0.3 s on the GloMax Navigator luminometer and then the luciferase activity of the prepared plate was measured.

20 μL of the stop glo luciferase assay reagent from the Dual-glo kit was dispensed into the measured plate, the device was placed at room temperature for 10 minutes and then the plate was shaken for 1 minute.

The luciferase activity was measured using a GloMax Navigator luminometer.

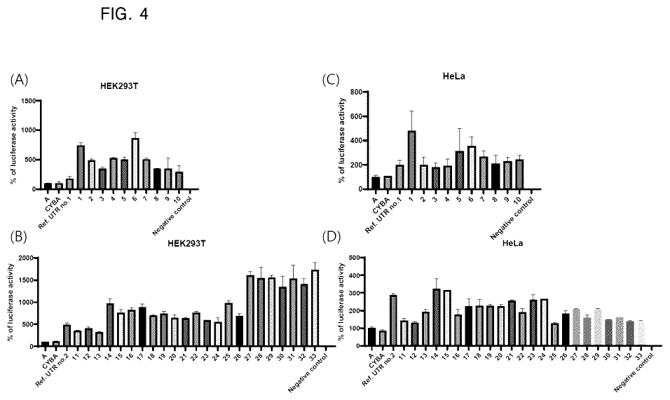

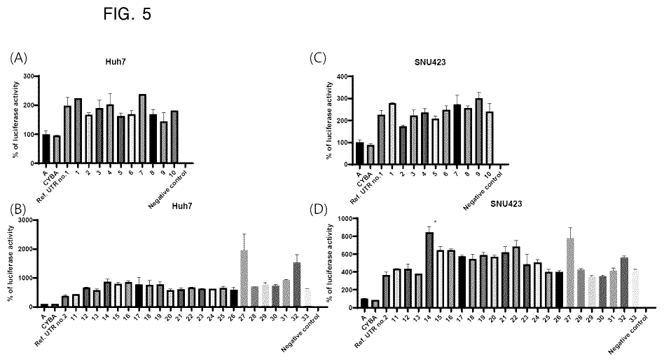

As a result, as can be seen from FIGS. 4 and 5, the translation efficiency of the mRNA including the UTR of the present invention was much superior to that of the control in all cell lines.

Although specific configurations of the present invention have been described in detail, those skilled in the art will appreciate that this detailed description is provided as preferred embodiments for illustrative purposes and should not be construed as limiting the scope of the present invention. Therefore, the substantial scope of the present invention is defined by the accompanying filed claims and equivalents thereto.

### INDUSTRIAL APPLICABILITY

The 5′-UTR polynucleotide according to the present invention can effectively induce the expression of a target protein due to improved translation efficiency thereof and thus is useful for various RNA-based applications, for example, vaccines, in vivo/ex vivo gene therapy, etc.

1. An isolated 5′-untranslated region (UTR) polynucleotide comprising a nucleotide sequence represented by a nucleic acid sequence of Formula (I) below:
AG[N₂₂]GCCACC. Formula (I):

2. An isolated 5′-untranslated region (UTR) polynucleotide comprising a nucleotide sequence represented by a nucleic acid sequence of Formula (II) below:
AGGA[N₁₉]RGCCACC (SEQ ID NO: 38) Formula (II):
wherein R represents A or G.

3. The isolated 5′-untranslated region (UTR) polynucleotide according to claim 1 or 2, wherein the 5′-UTR is any one of the nucleotide sequences represented by SEQ ID NOs: 1 to 33.

4. A synthetic nucleic acid molecule, in an order of 5′ to 3′, comprising:
a) a 5′-CAP structure:
b) the 5′-UTR polynucleotide according to claim 1 or 2:
c) at least one coding region:
d) a 3′-untranslated region (3′-UTR); and
e) 10 to 1,000 poly (A) tails or poly (A) tail-like sequences.

5. The synthetic nucleic acid molecule according to claim 4, wherein the 5′-CAP structure is selected from the group consisting of m⁷GpppAₘpG, m⁷GpppApG, and m7,3′OmeApppG.

6. The synthetic nucleic acid molecule according to claim 4, wherein the coding region encodes at least one protein selected from the group consisting of antigenic proteins, allergenic proteins, therapeutic proteins, and fragments, mutants or derivatives of the proteins.

7. The synthetic nucleic acid molecule according to claim 6, wherein the antigenic protein comprises at least one selected from the group consisting of tumor antigens, pathogenic antigens, autoantigens, alloantigens and allergens.

8. The synthetic nucleic acid molecule according to claim 7, wherein the tumor antigen is selected from the group consisting of NYESO-1, HER-2/neu, MAGE-1, tyrosinase, MUCI, CEA, Mam-A, hTERT, Syalyl-Tn, WT1, alpha-fetoprotein, CA-125, gp-100, p53, Ras, Src, EGFRVIII, PSMA, GD2, Bcr-abl, survivin, PSA, EphA2, PAP, AFP, EpCAM, ALK, mesothelin, PSCA, MART-1, Melan-A, SCP-1, SPAG9, AKAP4, and OY-TES-1.

9. The synthetic nucleic acid molecule according to claim 7, wherein the pathogenic antigen is selected from the group consisting of bacterial, viral, fungal and protist antigens.

10. The synthetic nucleic acid molecule according to claim 9, wherein the virus is a corona virus.

11. The synthetic nucleic acid molecule according to claim 4, wherein the poly (A) tail-like sequence has a configuration in which at least one nucleotide other than adenine selected from the group consisting of uracil (U), cytosine (C) and guanine (G) is inserted between a plurality of adenines or at an end of the poly (A) tail.

12. The synthetic nucleic acid molecule according to claim 4, wherein the synthetic nucleic acid molecule is RNA.

13. The synthetic nucleic acid molecule according to claim 12, wherein the RNA is selected from the group consisting of mRNA, viral RNA, self-replicating RNA and replicon RNA.

14. The synthetic nucleic acid molecule according to claim 4, wherein the synthetic nucleic acid molecule comprises at least one backbone-modified, sugar-modified or base-modified nucleic acid.

15. The synthetic nucleic acid molecule according to claim 4, wherein the 3′-UTR is selected from the group consisting of β-globin 3′-UTR, CYBA 3′-UTR, albumin 3′-UTR, growth hormone (GH) 3′-UTR, VEEV 3′-UTR, hepatitis B virus (HBV) 3′-UTR, α-globin 3′-UTR, DEN 3′-UTR, Barley Yellow Dwarf Virus-PAV (BYDV-PAV) 3′-UTR, elongation factor 1 α1 (EEF1A1) 3′-UTR, manganese peroxide dismutase (MnSOD) 3′-UTR, β subunit (β-mRNA) 3′-UTR of mitochondrial H(+)-ATP synthase, GLUT1 3′-UTR, MEF2A 3′-UTR, and β-F1-ATPase 3′-UTR.

16. A vaccine composition comprising the synthetic nucleic acid molecule according to claim 4.

17. The vaccine composition according to claim 16, wherein the synthetic nucleic acid (RNA) molecule is complexed or associated with one or more lipids to form one or more lipid nanoparticles or liposomes.

18. The vaccine composition according to claim 16, further comprising one or more adjuvants or active agents.


SEQUENCE LISTING (38 sequences)

SEQ ID NO: 1 - RNA, 30 nt, synthetic construct
  source 1..30: mol_type = other RNA
aggagaagagagaaaagtaaaatagccacc

SEQ ID NO: 2 - RNA, 30 nt, synthetic construct
  source 1..30: mol_type = other RNA
aggagtaaaagtaaaatagtaaaagccacc

SEQ ID NO: 3 - RNA, 30 nt, synthetic construct
  source 1..30: mol_type = other RNA
aggagaaaagaaagagtaaaataggccacc

SEQ ID NO: 4 - RNA, 30 nt, synthetic construct
  source 1..30: mol_type = other RNA
aggattaaaaatagaaagaataaggccacc

SEQ ID NO: 5 - RNA, 30 nt, synthetic construct
  source 1..30: mol_type = other RNA
aggacaacaaacaaaaacaaactagccacc

SEQ ID NO: 6 - RNA, 30 nt, synthetic construct
  source 1..30: mol_type = other RNA
aggacagagcactagaacaaaacagccacc

SEQ ID NO: 7 - RNA, 30 nt, synthetic construct
  source 1..30: mol_type = other RNA
aggaccactctcaaccaaaacctagccacc

SEQ ID NO: 8 - RNA, 30 nt, synthetic construct
  source 1..30: mol_type = other RNA
aggagacactaacaccccgaccgagccacc

SEQ ID NO: 9 - RNA, 30 nt, synthetic construct
  source 1..30: mol_type = other RNA
aggaccggaacacaaaaactagcagccacc

SEQ ID NO: 10 - RNA, 30 nt, synthetic construct
  source 1..30: mol_type = other RNA
aggaactacactagccactaaacagccacc

SEQ ID NO: 11 - RNA, 30 nt, synthetic construct
  source 1..30: mol_type = other RNA
aggacaactagacagagacaccgagccacc

SEQ ID NO: 12 - RNA, 30 nt, synthetic construct
  source 1..30: mol_type = other RNA
aggacacaacacccagaccaccgagccacc

SEQ ID NO: 13 - RNA, 30 nt, synthetic construct
  source 1..30: mol_type = other RNA
aggacacaactaccgaactactcagccacc

SEQ ID NO: 14 - RNA, 30 nt, synthetic construct
  source 1..30: mol_type = other RNA
aggagaactagtacccacccgacagccacc

SEQ ID NO: 15 - RNA, 30 nt, synthetic construct
  source 1..30: mol_type = other RNA
aggagagaaaaaaacaactacacagccacc

SEQ ID NO: 16 - RNA, 30 nt, synthetic construct
  source 1..30: mol_type = other RNA
aggagaaacaactaggaccacagagccacc

SEQ ID NO: 17 - RNA, 30 nt, synthetic construct
  source 1..30: mol_type = other RNA
aggacctaaaacacctcgacagcagccacc

SEQ ID NO: 18 - RNA, 30 nt, synthetic construct
  source 1..30: mol_type = other RNA
aggaaaaaactcacctctcagacagccacc

SEQ ID NO: 19 - RNA, 30 nt, synthetic construct
  source 1..30: mol_type = other RNA
aggagagaaactacacagactagagccacc

SEQ ID NO: 20 - RNA, 30 nt, synthetic construct
  source 1..30: mol_type = other RNA
aggaacaacagactcactcactcagccacc

SEQ ID NO: 21 - RNA, 30 nt, synthetic construct
  source 1..30: mol_type = other RNA
aggaaaactaaacagaacctaacagccacc

SEQ ID NO: 22 - RNA, 30 nt, synthetic construct
  source 1..30: mol_type = other RNA
aggaacagagaacactagagagcagccacc

SEQ ID NO: 23 - RNA, 30 nt, synthetic construct
  source 1..30: mol_type = other RNA
aggaccacacagacccacctactagccacc

SEQ ID NO: 24 - RNA, 30 nt, synthetic construct
  source 1..30: mol_type = other RNA
aggaaaccgactaaaaaccctctagccacc

SEQ ID NO: 25 - RNA, 30 nt, synthetic construct
  source 1..30: mol_type = other RNA
aggaacaacaccagcagaactcgagccacc

SEQ ID NO: 26 - RNA, 30 nt, synthetic construct
  source 1..30: mol_type = other RNA
aggaacaacagtcgtaaccctctagccacc

SEQ ID NO: 27 - RNA, 30 nt, synthetic construct
  source 1..30: mol_type = other RNA
aggaccctaaccaaacccactctagccacc

SEQ ID NO: 28 - RNA, 30 nt, synthetic construct
  source 1..30: mol_type = other RNA
aggacacaactctacccgactacagccacc

SEQ ID NO: 29 - RNA, 30 nt, synthetic construct
  source 1..30: mol_type = other RNA
aggaacacagtcagcaccaacacagccacc

SEQ ID NO: 30 - RNA, 30 nt, synthetic construct
  source 1..30: mol_type = other RNA
aggacactcagacaacccacactagccacc

SEQ ID NO: 31 - RNA, 30 nt, synthetic construct
  source 1..30: mol_type = other RNA
aggacctcaaaaaaaaactctctagccacc

SEQ ID NO: 32 - RNA, 30 nt, synthetic construct
  source 1..30: mol_type = other RNA
aggacaacagctcacacccccctagccacc

SEQ ID NO: 33 - RNA, 30 nt, synthetic construct
  source 1..30: mol_type = other RNA
aggacccagaacccacctaacagagccacc

SEQ ID NO: 34 - DNA, 37 bp, synthetic construct
  source 1..37: mol_type = other DNA
agtcttctggtccccacagactcagagagaacccacc

SEQ ID NO: 35 - DNA, 40 bp, synthetic construct
  source 1..40: mol_type = other DNA
agtgcgcgcctagcagtgtcccagccgggttcgtgtcgcc

SEQ ID NO: 36 - DNA, 47 bp, synthetic construct
  source 1..47: mol_type = other DNA
aggaaataagagagaaaagaagagtaagaagaaatataagagccacc

SEQ ID NO: 37 - DNA, 57 bp, synthetic construct
  source 1..57: mol_type = other DNA
gggaaataagagagaaaagaagagtaagaagaaatataagaccccggcgccgccacc

SEQ ID NO: 38 - RNA, 30 nt, synthetic construct
  source 1..30: mol_type = other RNA
aggannnnnnnnnnnnnnnnnnnrgccacc